SEQUENCE LISTING (40 sequences)

SEQ ID NO: 1 - DNA, 24 bp, Artificial sequence
  Synthetic DNA forward primer for -1023 SNP.
cctaaagtacttgacagcgagtgt

SEQ ID NO: 2 - DNA, 21 bp, Artificial sequence
  Synthetic DNA reverse primer for -1023 SNP.
gcacaggaggtgacttcaaca

SEQ ID NO: 3 - DNA, 17 bp, Artificial sequence
  Synthetic DNA VIC probe sequence for -1023 SNP.
ctgaggaaatcggcagc

SEQ ID NO: 4 - DNA, 17 bp, Artificial sequence
  Synthetic DNA FAM probe sequence for -1023 SNP.
ctgaggaaatcagcagc

SEQ ID NO: 5 - DNA, 22 bp, Artificial sequence
  Synthetic DNA forward primer for -654 SNP.
gtctatggctgtggttcggtat

SEQ ID NO: 6 - DNA, 23 bp, Artificial sequence
  Synthetic DNA reverse primer for -654 SNP.
cgcacatacaggcacaaatacac

SEQ ID NO: 7 - DNA, 17 bp, Artificial sequence
  Synthetic DNA VIC probe sequence for -654 SNP.
cagacatgctcagactt

SEQ ID NO: 8 - DNA, 17 bp, Artificial sequence
  Synthetic DNA FAM probe sequence for -654 SNP.
cagacatgcttagactt

SEQ ID NO: 9 - DNA, 20 bp, Artificial sequence
  Synthetic DNA forward primer for -468 SNP.
gccacagaagagccaaaagc

SEQ ID NO: 10 - DNA, 18 bp, Artificial sequence
  Synthetic DNA reverse primer for -468 SNP.
ccccagagggctaaagct

SEQ ID NO: 11 - DNA, 16 bp, Artificial sequence
  Synthetic DNA VIC probe sequence for -468 SNP.
ctggtaagcacaccac

SEQ ID NO: 12 - DNA, 16 bp, Artificial sequence
  Synthetic DNA FAM probe sequence for -468 SNP.
ctggtaaggacaccac

SEQ ID NO: 13 - DNA, 16 bp, Artificial sequence
  Synthetic DNA forward primer for -406 SNP.
gggtagccgggaagca

SEQ ID NO: 14 - DNA, 17 bp, Artificial sequence
  Synthetic DNA reverse primer for -406 SNP.
ccctcgccctccttctc

SEQ ID NO: 15 - DNA, 14 bp, Artificial sequence
  Synthetic DNA VIC probe sequence for -406 SNP.
tggtggcccgccct

SEQ ID NO: 16 - DNA, 14 bp, Artificial sequence
  Synthetic DNA FAM probe sequence for -406 SNP.
tggtggcctgccct

SEQ ID NO: 17 - DNA, 18 bp, Artificial sequence
  Synthetic DNA forward primer for -367 SNP.
ccctccagggagcagttg

SEQ ID NO: 18 - DNA, 18 bp, Artificial sequence
  Synthetic DNA reverse primer for -367 SNP.
ggcactcctcccctttcc

SEQ ID NO: 19 - DNA, 14 bp, Artificial sequence
  Synthetic DNA VIC probe sequence for -367 SNP.
cagccccaggagaa

SEQ ID NO: 20 - DNA, 15 bp, Artificial sequence
  Synthetic DNA FAM probe sequence for -367 SNP.
ccagcctcaggagaa

SEQ ID NO: 21 - DNA, 19 bp, Artificial sequence
  Synthetic DNA forward primer for -47 SNP.
ccgctgaatgaggcttcca

SEQ ID NO: 22 - DNA, 16 bp, Artificial sequence
  Synthetic DNA reverse primer for -47 SNP.
ccatggcgcgcagtct

SEQ ID NO: 23 - DNA, 14 bp, Artificial sequence
  Synthetic DNA VIC probe sequence for -47 SNP.
cctcagcgggcgga

SEQ ID NO: 24 - DNA, 14 bp, Artificial sequence
  Synthetic DNA FAM probe sequence for -47 SNP.
cctcagcaggcgga

SEQ ID NO: 25 - DNA, 16 bp, Artificial sequence
  Synthetic DNA forward primer for 46 SNP.
cggcagcgccttcttg

SEQ ID NO: 26 - DNA, 17 bp, Artificial sequence
  Synthetic DNA reverse primer for 46 SNP.
tgcgtgacgtcgtggtc

SEQ ID NO: 27 - DNA, 15 bp, Artificial sequence
  Synthetic DNA VIC probe sequence for 46 SNP.
cacccaatagaagcc

SEQ ID NO: 28 - DNA, 14 bp, Artificial sequence
  Synthetic DNA FAM probe sequence for 46 SNP.
acccaatggaagcc

SEQ ID NO: 29 - DNA, 20 bp, Artificial sequence
  Synthetic DNA forward primer for 79 SNP.
ccttcttgctggcacccaat

SEQ ID NO: 30 - DNA, 16 bp, Artificial sequence
  Synthetic DNA reverse primer for 79 SNP.
tgcccaccacccacac

SEQ ID NO: 31 - DNA, 17 bp, Artificial sequence
  Synthetic DNA VIC probe sequence for 79 SNP.
tcgtccctttgctgcgt

SEQ ID NO: 32 - DNA, 17 bp, Artificial sequence
  Synthetic DNA FAM probe sequence for 79 SNP.
tcgtccctttcctgcgt

SEQ ID NO: 33 - DNA, 19 bp, Artificial sequence
  Synthetic DNA forward primer for 252 SNP.
actggcctgtgctgatctg

SEQ ID NO: 34 - DNA, 15 bp, Artificial sequence
  Synthetic DNA reverse primer for 252 SNP.
tgggcggccccaaag

SEQ ID NO: 35 - DNA, 16 bp, Artificial sequence
  Synthetic DNA VIC probe sequence for 252 SNP.
accactgctaggccca

SEQ ID NO: 36 - DNA, 14 bp, Artificial sequence
  Synthetic DNA FAM probe sequence for 252 SNP.
cactgccaggccca

SEQ ID NO: 37 - DNA, 22 bp, Artificial sequence
  Synthetic DNA forward primer for 523 SNP.
ccttcttgcccattcagatgca

SEQ ID NO: 38 - DNA, 22 bp, Artificial sequence
  Synthetic DNA reverse primer for 523 SNP.
gcatagcagttgatggcttcct

SEQ ID NO: 39 - DNA, 15 bp, Artificial sequence
  Synthetic DNA VIC probe sequence for 523 SNP.
ctggtacagggccac

SEQ ID NO: 40 - DNA, 14 bp, Artificial sequence
  Synthetic DNA FAM probe sequence for 523 SNP.
tggtaccgggccac

(19) United States
(12) Patent Application Publication
(10) Pub. No.: US 2006/0069074 A1
(43) Pub. Date: Mar. 30, 2006

(54) Genetic predictor of efficacy of anti-asthmatic agents for improving pulmonary function

(76) Inventors: Robert F. Lemanske JR., Madison, WI (US); Christine A. Sorkness, Madison, WI (US); Vernon M. Chinchilli, Elizabethtown, PA (US); Wenlei Liu, Palmyra, PA (US); Brenda Phillips, Elizabethtown, PA (US); Robert S. Zeiger, San Diego, CA (US); Gregory P. Heldt, (US); Fernando D. Martinez, Tucson, AZ (US); Walter T. Klimecki, Vail, AZ (US); Theresa W. Guilbert, Tucson, AZ (US); Wayne J. Morgan, Tucson, AZ (US); Stanley J. Szefler, Aurora, CO (US); Gary L. Larsen, Littleton, CO (US); Lynn M. Taussig, Greenwood Village, CO (US); Joseph D. Spahn, Denver, CO (US); Robert C. Strunk, St. Louis, MO (US); Leonard B. Bacharier, Town & Country, MO (US); Gordon R. Bloomberg, St. Louis, MO (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 11/176,026

(22) Filed: Jul. 7, 2005

Related U.S. Application Data
(60) Provisional application No. 60/585,872, filed on Jul. 7, 2004.

(51) Int. Cl. A61K 31/56; A61K 31/47

(52) U.S. Cl. 514/171; 514/311

(57) ABSTRACT

The present invention relates to methods for determining and predicting the efficacy of therapeutics for the treatment of an individual with asthma based on that individual's β₂-adrenergic receptor. The invention finds particular applicability to the treatment of an individual with asthma with an inhaled corticosteroid or a leukotriene receptor antagonist who is homozygous for arginine at position 16 of the β₂-adrenergic receptor.

### CROSS-REFERENCE TO RELATED APPLICATIONS

This application claims the benefit of previously filed U.S. Provisional Patent Application No. 60/585,872, filed Jul. 7, 2004, incorporated herein by reference as if set forth in its entirety.

### STATEMENT REGARDING FEDERALLY SPONSORED RESEARCH OR DEVELOPMENT

This invention was made with United States government support awarded by the following agencies: NIH HL064305. The United States has certain rights in this invention.

### BACKGROUND

The invention relates generally to methods of treating an individual with asthma, and more particularly to genotypic methods of predicting the outcome of treating an individual having asthma.

Asthma is a chronic lung condition, in which airflow into and out of the lungs is restricted. Asthma occurs in 3% to 5% of the population at some time in life. Although asthma affects people of all ages, it often starts in childhood and is more common in children than in adults. Additionally, more young males have asthma than young females; however, in adulthood, more females have asthma than males. Furthermore, asthma is a problem among all ethnic groups, although individuals of African decent have slightly more asthma attacks than other ethnic groups and are slightly more likely than other ethnic groups to be hospitalized for asthma attacks.

Two physiologic events occur in asthma—bronchoconstriction and inflammation. First, the muscles of the bronchial tree constrict, which reduces airflow. Additionally, the cells of the bronchial tree secrete histamine, resulting in the secretion of copious amounts of mucus. The mucus further reduces airflow and also creates a barrier to efficient gas exchange in the lungs. Finally, the cells of the bronchial tree become inflamed, which also contributes to the reduced airflow. Thus, the hallmarks of asthma include, but are not limited to, wheezing, coughing, chest tightness and trouble breathing (dsypnea).

Clinicians have long known that asthma is not a single disease; it exists in many forms. Some forms of asthma are due to environmental factors, while other forms of asthma are due to genetic factors or are due to a combination of both. Environmental factors that can cause an asthma attack include, but are not limited to, the following: dust, animal dander, cockroaches, pollen, mold, infection, cold air, exertion, reactions to medications, chemicals and cigarette smoke. Genetic factors are discussed in greater detail below.

Although there is no cure for asthma yet, it can be controlled through medical treatment, as well as management of exposure to environmental factors. Medical treatment from asthma is through therapeutics such as anti-inflammatory agents and bronchodilators.

Anti-inflammatory agents are an important type of therapy for most individuals with asthma because these drugs prevent asthma on an long-term basis. One class of anti- inflammatory agents includes corticosteroids. Corticosteroids inhibit the production of leukotrienes and prostaglandins through interfering with arachidonic acid metabolism, through reducing the migration and the inhibition of the activity of inflammatory cells, and through enhancing of the responsiveness of β-adrenergic receptors in bronchial smooth muscle. Corticosteroids include budesonide (Pulmicort®), fluticasone (Flovent®), flunisolide (Aerobid(®), triamcinolone (Azmacort®), beclomethasone (Qvar®), ciclesonide (Alvesco®) and mometasone (Nasonex®).

A second class of anti-inflammatory agents is leukotriene receptor antagonists (LTRAs). LTRAs are non-steroidal, but are nonetheless anti-inflammatory agents. LTRAs block the action or inhibit the synthesis of the leukotrienes, which are bioactive mediators with proinflammatory effects that play an important role in the pathophysiology of asthma. Leukotrienes cause bronchoconstriction, mucus secretion, increased vascular permeability, and eosinophil migration to the airways, as well as promote smooth muscle cell proliferation. Although not a preferred first choice therapy, LTRAs can be given when a corticosteroid cannot, or will not, be used, or if the dose cannot be increased. LTRAs include montelukast (Singulair®), zafirlukast (Accolate®) and pranlukast (Alzaire®).

Clinical and physiological responses to ICS and to LTRAs vary significantly between subjects with asthma. Genetic factors may be important contributors to this variability. Malmstrom K, et al., Oral montelukast, inhaled beclomethasone, and placebo for chronic asthma. A randomized, controlled trial Montelukast/Beclomethasone Study Group, Ann. Intern. Med. 130:487-495 (1999); and Palmer L, et al., Pharmacogenetics of asthma, Am. J. Respir. Crit. Care Med. 165:861-866 (2002). Inconsistent responses are especially observed for LTRAs as compared with those to ICS, but the determinants of this unpredictability are not understood. Wenzel S, The role of leukotrienes in asthma, Prostaglandins Leukot. Essent. Fatty Acids 69:145-155 (2003).

Recently, a field of study known as pharmacogenomics, has been developed to study how genetic inheritance affects an individual's response to a drug. Pharmacogenomics combines traditional pharmaceutical sciences such as biochemistry with an understanding of common DNA variations in the human genome. The most common variations in the human genome are called single nucleotide polymorphisms (SNPs). There is estimated to be approximately 11 million SNPs in the human population, with an average of one SNP every 1,300 base pairs (bp).

Drysdale, C. et al., Complex promoter and coding region β2-adrenergic receptor haplotypes alter receptor expression and predict in vivo responsiveness, P.N.A.S. U.S.A. 97:10483-10488 (2000), incorporated by reference herein as if set forth in its entirety, characterized SNPs in the β₂-adrenergic receptor (B2AR) gene (chromosome 5q31-33). B2AR, a drug target for both long- and short-acting adrenergic bronchodilators, is a G protein-coupled receptor that causes bronchial smooth muscle to relax (bronchodilation) upon stimulation in the bronchial tree. Drysdale et al. also identified thirteen SNPs in the B2AR gene (at −1023 bp, −709 bp, −654 bp, −468 bp, −406 bp, −367 bp, −47 bp, −20 bp, 46 bp, 79 bp, 252 bp, 491 bp and 523 bp, measured from the first nucleotide of the start codon) and observed a dozen haplotypes in humans of various ethnicity. The polymorphic bases at relevant positions in each haplotype are noted in Drysdale et al., who further identified eighteen haplotype pairs (diplotype) in a group of asthmatic individuals, with over 85% of the asthmatic individuals belonging to one of five haplotype pairs.

The predictive value of the five most common asthma-associated diplotypes was determined by assessing the bronchodilator response to albuterol, a β-agonist. Drysdale et al. found that diplotype was significantly related to improvements in forced expiratory volume in one second (FEV₁) to albuterol. Diplotype 4/6 had the highest response; conversely, diplotype 4/4 had the lowest response. Drysdale et al., however, neither examined nor predicted whether asthmatic individuals having diplotype 4/4 would respond as poorly or better to any other asthma treatment.

Diplotype 4/4 contains a widely studied SNP because approximately one human in seven has the 4/4 diplotype at the B2AR gene. The SNP of interest at nucleotide 46 of haplotype 4 is an A that results in a glycine-to-arginine substitution at amino acid residue 16 in B2AR. Individuals with diplotype 4/4, as well as several other diplotypes, are homozygous for arginine at residue 16 in the B2AR.

Polymorphisms in the gene coding for the B2AR are among the most widely studied variants with a potential role in the pharmacogenetics of asthma. Palmer L, supra, and Ober C & Moffatt M, Contributing factors to the pathobiology. The genetics of asthma, Clin. Chest. Med. 21:245-261 (2002). Functionally, the Arg16 allele is associated with less down-regulation of agonist-exposed B2AR than the Gly16 allele, and studies of bronchial responses to albuterol in subjects who were naïve to B2AR-agonists suggested that carriers of the Arg16 allele showed higher immediate responses to albuterol than carriers of the Gly16 allele. Green S, et al., Amino-terminal polymorphisms of the human beta 2-adrenergic receptor impart distinct agonist-promoted regulatory properties, Biochemistry 33:9414-9419 (1994); and Martinez F, et al., Association between genetic polymorphisms of the beta 2-adrenoceptor and response to albuterol in children with and without a history of wheezing, J. Clin. Invest. 100:3184-3188 (1997); and Lima J, et al., Impact of genetic polymorphisms of the beta2-adrenergic receptor on albuterol bronchodilator pharmacodynamics, Clin. Pharmacol. Ther. 65:519-525 (1999). However, clinical trials evaluating subjects with asthma have consistently shown that homozygotes for Arg16 who are treated with regularly scheduled albuterol have significant deterioration in lung function as compared with carriers of the other two genotypes. Israel E, et al., The effect of polymorphisms of the beta(2)-adrenergic receptor on the response to regular use of albuterol in asthma, Am. J. Respir. Crit. Care Med. 162:75-80 (2000).

To explain these findings, it was initially proposed that Arg16 homozygotes were more likely to develop B2AR down-regulation while on regularly scheduled B2AR agonists. Liggett S, Polymorphisms of the beta2-adrenergic receptor and asthma, Am. J. Respir. Crit. Care Med. 156:S156-S162 (1997). However, more recent studies using genetically modified mice have generated an alternative hypothesis: persistent activation of B2AR does induce bronchodilation by Gs-protein-mediated mechanisms, but can also induce concomitant sensitization of Gq-protein coupled receptors that exert a bronchoconstrictive effect, including the receptor for cysteinyl leukotrienes. McGraw D, et al., Antithetic regulation by beta-adrenergic receptors of Gq receptor signaling via phospholipase C underlies the airway beta-agonist paradox, J. Clin. Invest. 112:619-626 (2003).

Recently, Litonjua et al also reported increased responses to methacholine among Arg16 homozygotes. Litonjua A, et al., Beta 2-adrenergic receptorpolymorphisms and haplotypes are associated with airways hyper-responsiveness among nonsmoking men, Chest 126:66-74 (2004). Interestingly, some patients treated chronically with β-agonists show paradoxical deterioration in lung function and increased bronchial responsiveness. Israel E, et al., supra; and Cheung D, et al., Long-term effects of a long-acting beta 2-adrenoceptor agonist, salmeterol, on airway hyper-responsiveness in patients with mild asthma, N. Engl. J. Med. 327:1198-1203 (1992).

### SUMMARY

In one aspect, the invention relates to a method is for predicting a negative pulmonary response to asthma therapy with a leukotriene receptor antagonist (LTRA) in an individual having a B2AR that is homozygous for arginine at B2AR residue 16 and having a baseline FEV₁. One tests the individual to see if he or she exhibits a positive pulmonary response to asthma therapy with an inhaled corticosteroid (ICS). The positive pulmonary response with the ICS is correlated with the negative pulmonary response of the LTRA such that the positive pulmonary response is predictive of the negative pulmonary response.

In one embodiment, the negative pulmonary response is characterized as a deterioration in FEV₁ with the LTRA relative to the baseline FEV₁ of the subject.

In another embodiment, the ICS is fluticasone propionate, beclomethasone dipropionate, budesonide, triamcinalone acetonide, flunisolide, ciclesonide or mometasone.

In yet another embodiment, the LTRA is montelukast sodium, zafirlukast or pranlukast.

In still another embodiment, the ICS is fluticasone propionate and the LTRA is montelukast sodium.

In a second aspect, the invention relates to a method for avoiding deterioration of FEV₁ in an individual having a B2AR that is homozygous for arginine at residue 16 and having a baseline FEV₁. First, one tests the individual to determine if he or she exhibits a strong positive pulmonary response to therapy with an ICS. The strong positive pulmonary response is characterized as an increase in FEV₁ of at least about 10% over the baseline FEV₁ of the individual. Next, one treats the individual having a strong positive response with an anti-asthmatic agent other than an LTRA.

In an embodiment, the anti-asthmatic agent is an ICS.

In another embodiment, the ICS is fluticasone propionate, beclomethasone dipropionate, budesonide, triamcinalone acetonide, flunisolide, ciclesonide or mometasone.

In still another embodiment, the method includes establishing a negative correlation between a pulmonary response to asthma therapy with an ICS and a pulmonary response to asthma therapy with an LTRA in an individual having a B2AR that is homozygous for arginine at residue 16. In accord with the method, one measures a baseline pulmonary response for a number of individuals who have asthma. One treats each individual with a pair of anti-asthma drugs. Each drug is administered separately for a clinically relevant period in a randomized crossover treatment sequence. The anti-asthmatic drugs can be an ICS and an LTRA. One determines a change in response from the baseline pulmonary response for each individual after each treating step. A positive pulmonary response is an increase from the baseline and a negative pulmonary response is a response that is not a positive pulmonary response and assigns each individual to a group that is defined by the amino acid encoded at residue 16 of the B2AR, with at least one group containing individuals homozygous for arginine at residue 16. One plots a regression plot between the responses to the drugs for each group and identifies in the regression plot a negative correlation between the pulmonary response to the drugs in the individuals homozygous for arginine at residue 16 of the B2AR. The plot demonstrates that individuals homozygous for arginine at residue 16 exhibit a positive pulmonary response to the treatment with the ICS and a negative pulmonary response to the treatment with the LTRA.

In one embodiment, the measured pulmonary response is an FEV₁.

In another embodiment, the positive pulmonary response is an increase of at least about 7.5% of the baseline pulmonary response.

In a related embodiment, the response can be a measured time to first asthma exacerbation requiring oral prednisone treatment.

In still another embodiment, the ICS is fluticasone propionate, beclomethasone dipropionate, budesonide, triamcinalone acetonide, flunisolide, ciclesonide or mometasone.

In a further embodiment, the LTRA is montelukast sodium, zafirlukast or pranlukast.

In yet another embodiment, the ICS is fluticasone propionate and the LTRA is montelukast sodium.

It is a first advantage that the method identifies a treatment for individuals with the 4/4 diplotype.

It is also an advantage that the method permits individuals with the 4/4 diplotype to avoid LTRA therapy, to which they are unresponsive.

These and other features, aspects and advantages of the present invention will become better understood with reference to the following description and appended claims.

### BRIEF DESCRIPTION OF THE DRAWINGS

FIG. 1 shows the estimated least squares curves for the association between FEV₁ responses to montelukast and fluticasone in participants with different haplotype combinations (diplotypes) for the B2AR gene.

FIG. 2 depicts in separate panels, survival plots of individuals having distinct genotypes (A/A, A/G, and G/G) that encode amino acid 16 of B2AR in three treatment regimens.

### DETAILED DESCRIPTION OF A PREFERRED EMBODIMENT

As used herein, a haplotype is defined as a specific combination of SNPs that are contained within the promoter region and/or the translated region of a gene.

As used herein, a diplotype or haplotype pair is defined as a specific combination of haplotypes.

As used herein, an individual homozygous for arginine at residue 16 of B2AR is defined as an individual whose nucleic acid includes an A at the polymorphic SNP at nucleotide 46 of the B2AR gene that results in a glycine-to-arginine substitution at amino acid residue 16 in the B2AR encodes arginine at residue 16 of B2AR. The homozygote can have diplotype 4/4 or another diplotype in the B2AR gene. For example, haplotypes 1, 9, and 13 also contain Arg16 (Table 1, infra), and participants could thus be homozygous for Arg16 without having the 4/4 diplotype.

As used herein, a forced expiratory volume in one second (FEV₁) is a volume in liters of air expired during first second of forced expiration.

The present invention will be better understood upon consideration of the following non-limiting examples.

### EXAMPLE 1

This example shows the effect of inhaled corticosteroids (ICS) and leukotriene receptor antagonists (LTRAs) in asthmatic individuals having diplotype 4/4 in the B2AR gene.

To address the determinants of responses to montelukast (an LTRA) as compared to those to fluticasone propionate (an ICS), we conducted a doubled-blind, randomized trial in which these medicines were administered to a group of over 140 children in a crossover design. Strunk R, et al., Relationship of exhaled nitric oxide to clinical and inflammatory markers of persistent asthma in children, J. Allergy Clin. Immunol. 112:883-892 (2003), incorporated herein by reference as if set forth in its entirety.

Briefly, 144 children ages 6 to 17 years with mild to moderate persistent asthma were enrolled. They had a combination of asthma symptoms or rescue bronchodilator use on average of 3 or more days per week during the previous 4 weeks; ≧12% improvement in FEV₁ following a maximal bronchodilator testing procedure with albuterol MDI and/or methacholine PC₂₀ ≦12.5 mg/ml; no corticosteroid treatment within 4 weeks prior to enrollment with the exception of regular use of nasal corticosteroids; no leukotriene modifier agents within 2 weeks; and no history of respiratory tract infection within 4 weeks of enrollment. Children with severe asthma were excluded. Strunk R, et al., supra. Informed consent, approved by the Institutional Review Board of the subject's respective study institution, was obtained from a parent/guardian.

After a 5 to 10 day characterization period, participants were randomized to one of two crossover treatment sequences with 8-week periods of either fluticasone propionate (Flovent Diskus®, GlaxoSmithKline 100 mcg per inhalation administered as one inhalation twice daily), or montelukast (Singulair®, Merck, 5 mg chewable tablet for those 6 to 14 years and 10 mg tablet for those 15 to 18 years of age taken as one tablet by mouth each night). During the active treatment period for one drug, the subject received a placebo for the alternative drug. Adherence to medication was objectively monitored. The two crossover sequences were stratified according to clinical center, age category (ages 6 to 9; 10 to 14; and 15 to 18 years), and FEV₁ % predicted category (FEV₁ <85%; FEV₁ ≧85%) by the minimization method of randomization. Pulmonary function, measured 8 weeks after the initiation of a treatment, was the primary outcome measure of this trial. The protocol was approved by NHLBI's Protocol Review Committee and Data and Safety Monitoring Board. Study medications and matching placebos were provided by Merck & Co., Inc. (West Point, Pa.) and Glaxo Smith Kline (Research Triangle Park, N.C.).

One striking result of this trial was that despite a significant concordance in the FEV₁ responses to both medicines, almost one-fourth of all children responded to fluticasone but not to montelukast, while only a small percent responded to montelukast but not to fluticasone. Szefler S, et al., Characterization of within-subject responses tofluticasone and montelukast in childhood asthma, J. Allergy Clin. Immunol. 115:233-242 (2005), incorporated herein by reference as if set forth in its entirety.

Using participants enrolled in the trial, we studied the role of haplotypes in the B2AR gene on responses to these two medicines. Genomic DNA was genotyped at ten of the 13 previously published polymorphic sites in B2AR. Drysdale C, et al., supra. The numbering system chosen was that of Drysdale et al. Id. Site −709 was excluded because it was reported to occur in <1% of Caucasians only. Site +491 was similarly excluded because it was reported to occur at a low frequency (<3% for all ethnic groups). Site −20 was excluded because it was reported as being in complete linkage disequilibrium in all ethnicities with site −47, and site −367 both of which were tested. Samples were genotyped using 5′ exonuclease (Taqman, Applied Biosystems) assays, with standard reaction conditions. All assays genotyped a high fraction of the total samples, with non-assignment rates ranging from 0.69% to 4.86%. Haplotypes were inferred from the unphased genotypes using a Gibbs-sampling approach, as implemented in the PHASE (Version 2.02) software program, using a recombination model, with 100 burn-in and main iterations, a thinning interval of 1, and output probability thresholds for genotypes and haplotypes of 0.9.

TABLE 1
Primer and Probe Sequences of Taqman Reaction.

| | | |
|---|---|---|
| SNP | | |
| Loci | Primer Sequence | Probe Sequence |

| | | |
|---|---|---|
| −1023 | F: 5′-CCTAAAGTACTTGACAGCGAGTGT-3′ | VIC: 5′-CTGAGGAAATCgGCAGC-3′ |
| | R: 5′-GCACAGGAGGTGACTTCAACA-3′ | FAM: 5′-CTGAGGAAATCaGCAGC-3′ |
| −654 | F: 5′-GTCTATGGCTGTGGTTCGGTAT-3′ | VIC: 5′-CAGACATGCTcAGACTT-3′ |
| | R: 5′-CGCACATACAGGCACAAATACAC-3′ | FAM: 5′-CAGACATGCTtAGACTT-3′ |
| −468 | F: 5′-GCCACAGAAGAGCCAAAAGC-3′ | VIC: 5′-CTGGTAAGcACACCAC-3′ |
| | R: 5′-CCCCAGAGGGCTAAAGCT-3′ | FAM: 5′-CTGGTAAGgACACCAC-3′ |
| −406 | F: 5′-GGGTAGCCGGGAAGCA-3′ | VIC: 5′-TGGTGGCCcGCCCT-3′ |
| | R: 5′-CCCTCGCCCTCCTTCTC-3′ | FAM: 5′-TGGTGGCCtGCCCT-3′ |
| −367 | F: 5′-CCCTCCAGGGAGCAGTTG-3′ | VIC: 5′-CAGCCcCAGGAGAA-3′ |
| | R: 5′-GGCACTCCTCCCCTTTCC-3′ | FAM: 5′-CCAGCCtCAGGAGAA-3′ |
| —47 | F: 5′-CCGCTGAATGAGGCTTCCA-3′ | VIC: 5′-CCTCAGCgGGCGGA-3′ |
| | R: 5′-CCATGGCGCGCAGTCT-3′ | FAM: 5′-CCTCAGCaGGCGGA-3′ |
| 46 | F: 5′-CGGCAGCGCCTTCTTG-3′ | VIC: 5′-CACCCAATaGAAGCC-3′ |
| | R: 5′-TGCGTGACGTCGTGGTC-3′ | FAM: 5′-ACCCAATgGAAGCC-3′ |
| 79 | F: 5′-CCTTCTTGCTGGCACCCAAT-3′ | VIC: 5′-TCGTCCCTTTgCTGCGT-3′ |
| | R: 5′-TGCCCACCACCCACAC-3′ | FAM: 5′-TCGTCCCTTTcCTGCGT-3′ |
| 252 | F: 5′-ACTGGCCTGTGCTGATCTG-3′ | VIC: 5′-ACCACTGCtAGGCCCA-3′ |
| | R: 5′-TGGGCGGCCCCAAAG-3′ | FAM: 5′-CACTGCcAGGCCCA-3′ |
| 523 | F: 5′-CCTTCTTGCCCATTCAGATGCA-3′ | VIC: 5′-CTGGTACaGGGCCAC-3′ |
| | R: 5′-GCATAGCAGTTGATGGCTTCCT-3′ | FAM: 5′-TGGTACcGGGCCAC-3′ |

Statistical Analyses: Haplotype and diplotype frequencies, as well as baseline summary statistics by diplotype, were summarized via PROC FREQ and PROC MEANS of SAS 8.2 (SAS Institute, Inc, Cary, N.C.), respectively. Response to medication was measured as the percent change in pre-bronchodilator FEV₁ percent predicted from baseline to the end of each treatment period. A mixed-effects linear model was applied to account for period and sequence effects within the repeated measurements feature of the crossover design for the FEV₁ response. Restricted maximum likelihood estimation was applied to estimate all of the model parameters via PROC MIXED of SAS 8.2 (SAS Institute, Inc, Cary, N.C.). Microsoft PowerPoint 2000 was used for graphical displays. Least squares lines were overlaid on the scatterplots to display linear relationships between the two responses. Pearson correlation coefficients were used for inferential purposes; hypothesis testing was performed using Fisher's Z transformation for each correlation. A 2-sided p-value <0.05 was considered significant.

Results: Of 144 children DNA was available for 142. None of the ten B2AR variants genotyped deviated significantly from Hardy-Weinberg equilibrium (data not shown). Of the 10 reported haplotypes that could have been identified with the 10 SNPs genotyped herein, 8 were found in this population, and one was novel (called haplotype 13, Table 2).

As shown in Table 2, there were significant differences in the distribution of the haplotypes among the three main ethnic groups (African-Americans, Caucasians, and Hispanics). Given the small numbers of participants with haplotypes other than 2, 4, and 6, participants that did not carry any of these 3 haplotypes were initially collapsed into a single group. The distribution of diplotypes in the population is reported in Table 3.

TABLE 2
SNPs tested and haplotypes identified for B2AR gene in the four ethnic groups.

| | | | | | | | | | | | His- | Cau- | African- | |
|---|---|---|---|---|---|---|---|---|---|---|---|---|---|---|
| Nucleotide | −1023 | −654 | −468 | −406 | −367 | −47 | 46 | 79 | 252 | 523 | panics | casians | Americans | Other |
| Chromosome | | | | | | | | | | | 68 | 150 | 60 | 6 |
| | | | | | | | | | | | chromo- | chromo- | chromo- | chromo- |
| | | | | | | | | | | | somes | somes | somes | somes |
| Alleles: | G/A | G/A | C/G | C/T | T/C | T/C | G/A | C/G | G/A | C/A | | | | |
| Haplotype | | | | | | | | | | | n (%) | n (%) | n (%) | n (%) |
| 1 | A | G | C | C | T | T | A | C | G | C | 4 (5.9) | 0 (0.0) | 6 (10.0) | 1 (16.7) |
| 2 | A | G | G | C | C | C | G | G | G | C | 16 (23.5) | 68 (45.3) | 9 (15.0) | 3 (50.0) |
| 4 | G | A | C | C | T | T | A | C | G | C | 24 (35.3) | 50 (33.3) | 17 (28.3) | 2 (33.3) |
| 6 | G | G | C | C | T | T | G | C | A | A | 22 (32.4) | 25 (16.7) | 20 (33.3) | 0 (0.0) |
| 7 | G | G | C | C | T | T | G | C | A | A | 0 (0.0) | 1 (0.7) | 0 (0.0) | 0 (0.0) |
| 9 | A | G | C | T | T | T | A | C | G | C | 0 (0.0) | 0 (0.0) | 8 (13.3) | 0 (0.0) |
| 10 | G | G | C | C | T | T | G | C | A | C | 2 (2.9) | 4 (2.7) | 0 (0.0) | 0 (0.0) |
| 11 | G | G | C | C | T | T | G | C | G | C | 0 (0.0) | 1 (0.7) | 0 (0.0) | 0 (0.0) |
| 13* | G | A | C | C | T | C | A | C | G | C | 0 (0.0) | 1 (0.7) | 0 (0.0) | 0 (0.0) |

*Novel haplotype

TABLE 3
Frequency of diplotypes for the B2AR gene in the population.

| Diplotype | n | % |
|---|---|---|
| 2/2 | 20 | 14.1 |
| 2/4 | 28 | 19.7 |
| 2/6 | 21 | 14.8 |
| 4/4 | 20 | 14.1 |
| 4/6 | 18 | 12.7 |
| 6/6 | 9 | 6.3 |
| All rare* | 26 | 18.3 |
| | 142 | 100.0 |

*Collapsing 13 other diplotypes

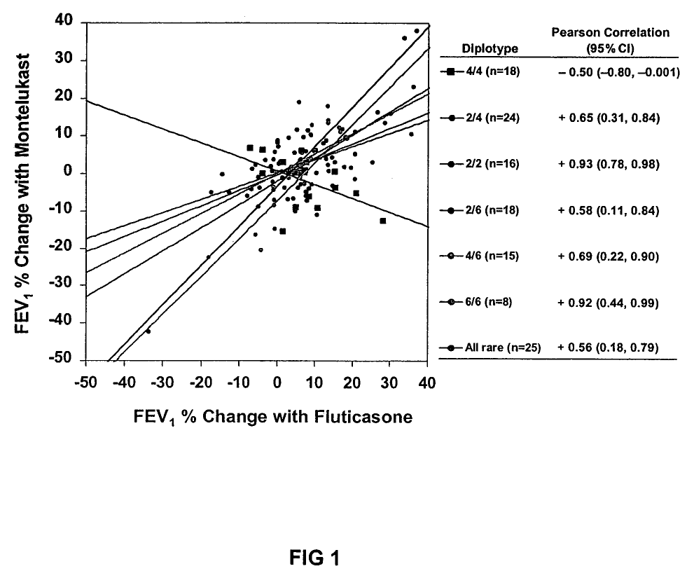

Eighteen participants did not complete all requirements of the trial for various reasons. Twelve of these met treatment failure criteria and did not complete both drug treatment periods; however, no diplotype was preferentially represented in this subset (data not shown). Plots for the regression between responses to montelukast and fluticasone for the 7 diplotype groups in the remaining 124 participants are shown in FIG. 1. Whereas significant positive Pearson's correlations between the responses to both medicines were found for diplotypes 2/2 (+0.93, n=16, p<0.0001), 2/4 (+0.65, n=24, p=0.0006), 2/6 (+0.58, n=18, p=0.011), 4/6 (+0.69, n=15, p=0.004), 6/6 (+0.92, n=8, 0.001), and all rare combined (+0.56, n=25, p=0.004), carriers of the 4/4 diplotype showed a significant negative correlation (−0.50, n=18, p=0.03) between responses to both treatments. FIG. 1 also shows that this negative correlation almost invariably meant that better observed responses to fluticasone were associated with worse observed responses to montelukast. This discrepancy in correlation coefficients among all groups was highly significant (F_{6.103}=40.13, p=<0.000001). Pearson's correlation for the montelukast vs. fluticasone regression curve for the combined non-4/4 diplotypes was +0.72, highly significantly different from that for the 4/4 diplotype (t=5.25, p=0.0000007). Similar patterns were observed in Caucasians, Hispanics, and African-Americans (not shown).

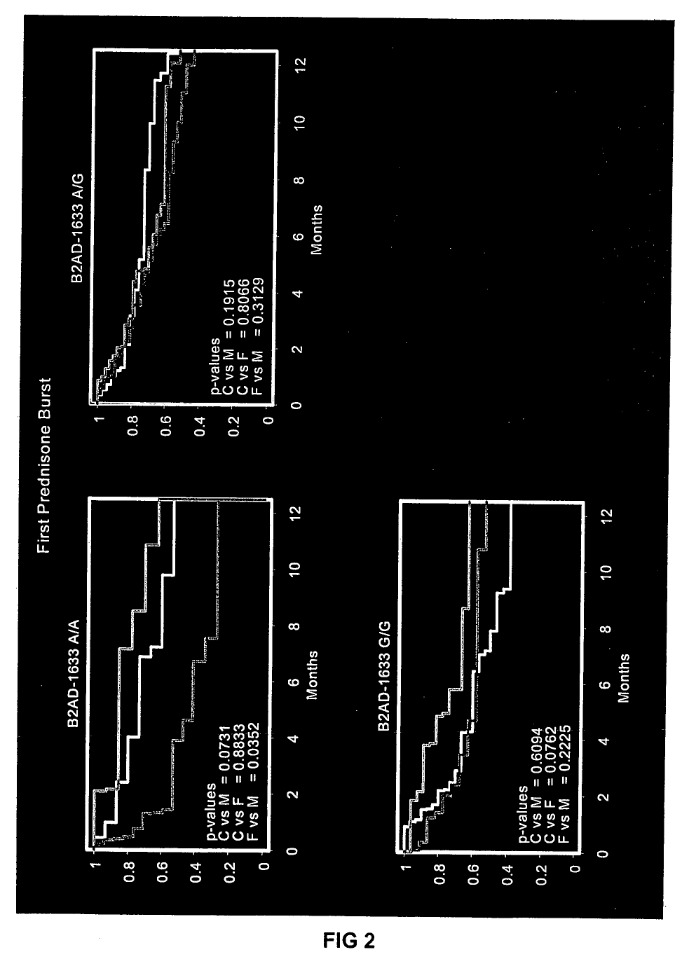

As shown in FIG. 2, we grouped all participants with non-4/4 diplotypes who were Arg/Arg homozygotes into one group (n=6), and plotted the regression curves for responses to both medicines in this group, the group with diplotype 4/4 and the group of other diplotypes combined. Individuals with diplotype 4/4 were the majority (75%) of Arg16 homozygotes. Arg16 homozygotes that belonged to diplotypes other than 4/4 had a positive correlation between responses to montelukast and fluticasone, suggesting that these participants responded in a manner similar to that of other non-4/4 diplotypes. However, the difference between the correlation coefficients for the non-4/4 Arg/Arg homozygotes and the 4/4 homozygotes did not reach statistical significance (t=1.34, p=0.20).

When compared with all other diplotypes, 4/4 diplotypes had higher mean percent predicted pre-bronchodilator FEV₁ and FEV₁/FVC ratio at baseline (p=0.048 and p=0.092, respectively, Table 4). The dose of methacholine causing a 20% fall in FEV₁ was 1.3 doubling doses lower in the 4/4 diplotype than in the other diplotypes, adjusting for baseline percent predicted FEV₁ (p==0.015). Exhaled nitric oxide at baseline was twice as high in children with the 4/4 diplotype as in children with the other diplotypes (p=0.01 1). As shown in Table 4, there were no significant differences in total eosinophil counts, serum eosinophilic cationic protein, urinary leukotrienes or albuterol use before or after randomization (data not shown) between groups.

TABLE 4
Mean (95% CI) baseline characteristics for the 4/4 diplotype and for all
others. Only the 124 genotyped participants who completed all study requirements were
included.

| | 4/4 | All Others | |
|---|---|---|---|
| | n = 18 | n = 106 | p-value |
| Baseline pre-bronchodilator FEV₁ % predicted | 101.4 (95.6, 107.1) | 94.7 (92.1, 97.3) | 0.048 |
| Baseline pre-bronchodilator FEV₁/FVC (%) | 83.0 (79.61, 86.4) | 79.6 (78.1, 81.1) | 0.092 |
| Methacholine PC₂₀ (mg/ml)*⁺ | 0.6 (0.3, 1.1) | 1.4 (1.1, 1.7) | 0.015 |
| Exhaled nitric oxide⁺ | 44.4 (25.9, 76.2) | 22.5 (18.5, 27.5) | 0.011 |
| Blood total eosinophil count (absolute) | 328.1 (213.4, 504.5) | 237.2 (202.5, 277.8) | 0.125 |
| (cells/mm³)⁺ | | | |
| Urinary Leukotrienes E4 (pg/mg creatinine)⁺ | 106.2 (86.7, 129.9) | 99.1 (90.0, 109.0) | 0.573 |
| Serum eosinophilic cationic protein (mcg/L)⁺ | 19.9 (12.3, 31.9) | 15.4 (13.2, 17.9) | 0.215 |

*Corrected for baseline percent predicted FEV₁
⁺Geometric means with 95% confidence limits are presented.

In this study we used a crossover approach to assess responses to the two most widely used asthma control medications—ICS and LTRAs. That is, instead of evaluating these responses in parallel samples, we tested responses consecutively and in random order in the same participant sample. We believe that this approach more faithfully reflects the therapeutic choices often made by physicians in clinical practice. We found that children with asthma with diplotype 4/4 for the B2AR gene showed a dramatically different pattern of relative FEV₁ responses to montelukast and fluticasone from that observed in the rest of the population. Changes in FEV₁ induced by these two medicines were positively correlated among most participants, but responses in 4/4 diplotypes were significantly, but negatively correlated. Graphical representation of the data revealed that, in almost all cases, this negative correlation indicated that higher responses to fluticasone were associated with lower responses to montelukast.

### EXAMPLE 2

A second study of the association between response to inhaled corticosteroids as compared with that to montelukast in individuals with different genotypes and diplotypes for the B2AR gene was recently performed. In this second clinical trial, responses to 3 different controller regimens were studied. These included: 100 micrograms fluticasone twice daily; combination of fluticasone 100_micrograms once daily plus salmuterol 50 micrograms twice daily; and montelukast 5 milligrams once daily. Children were treated with one of these three regimens for a 12 month period.

FIG. 2 shows survival analyses (that is, individuals not requiring prednisone up to the indicated given time point) for individuals having the Arg/Arg (B2AD-1633 A/A), Arg/Gly (B2AD-1633 A/G), and Gly/Gly (B2AD-1633 G/G) genotypes for the polymorphism at codon 16 of the B2AR gene. Carriers of the Arg/Arg genotype treated with montelukast showed a significantly shorter time to first asthma exacerbation requiring oral prednisone treatment (the lowest of three curves in the graph labeled ‘B2AD-1633 A/A’) as compared with children having the same genotype but who were treated with either of the other 2 regimens. Conversely, no significant difference in time to first asthma exacerbation requiring oral prednisone treatment was observed among the three regimens either among carriers of the Arg/Gly genotype or among carriers of the Gly/Gly genotype.

These results replicate the results of Example 1, and strongly indicate that carriers of the Arg/Arg genotype show not only worse lung function responses, but also worse clinical responses to montelukast as compared to inhaled corticosteroids. However, contrary to the results observed in Example 1, the same trends were observed among Arg/Arg individuals who carried the 4/4-haplotype and among Arg/Arg individuals who did not carry the 4/4-diplotype. More specifically, treatment with montelukast was associated with a shorter time to first asthma exacerbation both among Arg/Arg carriers of the 4/4-diplotype and among Arg/Arg carriers of other haplotype combinations, when compared with subjects with the same diplotype/haplotype combinations who were treated with either of the other 2 regimens.

Although this result was not observed for non-4/4-diplotype Arg/Arg carriers in the first Example, that Example may have lacked sufficient statistical power to observe an association between FEV₁ responses to fluticasone relative to those to montelukast among non-4/4 carriers of the Arg/Arg genotype. Alternatively, responses to therapy may show somewhat different relations to the different B2AR gene polymorphisms when the measured outcome is FEV₁ than when the measured outcome is exacerbation to first prednisone burst. Further study and data analysis is expected to confirm the association.

In either case, the observation associated with the Arg/Arg genotype is clinically important. These results have important implications for future asthma therapy: homozygotes for haplotype 4 of the B2AR gene, representing one out of seven children with asthma, have impaired FEV₁ responses to montelukast as compared to fluticasone. These pharmacogenetic effects may need to be considered when making therapeutic decisions about asthma control medications in these children.

Those of ordinary skill in the art will readily appreciate that the foregoing represents merely certain preferred embodiments of the invention. Various changes and modifications to the procedures and compositions described above can be made without departing from the spirit or scope of the present invention, as set forth in the claims below.

The invention claimed is:

1. A method for predicting a negative pulmonary response to therapy with a leukotriene receptor antagonist in a subject having a β₂ adrenergic receptor characterized as being homozygous for arginine at residue 16, the subject further having a baseline forced expiratory volume in one second (FEV₁), the method comprising the step of:
ascertaining that the subject exhibits a positive pulmonary response to therapy with an inhaled corticosteroid, the positive pulmonary response being correlated with the negative pulmonary response such that the positive pulmonary response is predictive of the negative pulmonary response.

2. The method as claimed in claim 1 wherein the positive pulmonary response is characterized as an increase in FEV₁ of at least about 7.5% over the baseline FEV₁ of the subject and the negative pulmonary response is characterized as a response that is not a positive pulmonary response.

3. The method as claimed in claim 1 wherein the negative pulmonary response is characterized as a deterioration in FEV₁ relative to the baseline FEV₁ of the subject.

4. The method as claimed in claim 1 wherein the inhaled corticosteroid is selected from the group consisting of fluticasone propionate, beclomethasone dipropionate, budesonide, triamcinalone acetonide, flunisolide, ciclesonide, and mometasone.

5. The method as claimed in claim 1 wherein the leukotriene receptor antagonist is selected from the group consisting of montelukast sodium, zafirlukast and pranlukast.

6. The method as claimed in claim 1 wherein the inhaled corticosteroid is fluticasone propionate and the leukotriene receptor antagonist is montelukast sodium.

7. A method for avoiding deterioration of forced expiratory volume in one second (FEV₁) in a subject having a β₂ adrenergic receptor characterized as being homozygous for arginine at residue 16, the subject further having a baseline FEV₁, the method comprising the step of:
ascertaining that the subject exhibits a strong positive pulmonary response to therapy with an inhaled corticosteroid, the strong positive pulmonary response being characterized as an increase in FEV₁ of at least about 10% over the baseline FEV₁ of the subject;
treating the subject with an anti-asthmatic agent other than a leukotriene receptor antagonist.

8. The method as claimed in claim 7 wherein the anti-asthmatic agent is an inhaled corticosteroid.

9. The method as claimed in claim 8 wherein the inhaled corticosteroid is selected from the group consisting of fluticasone propionate, beclomethasone dipropionate, budesonide, triamcinalone acetonide, flunisolide, ciclesonide, and mometasone.

10. A method for establishing a negative correlation between a pulmonary response to therapy with an inhaled corticosteroid and a pulmonary response to therapy with a leukotriene receptor antagonist in an individual characterized as being homozygous for arginine at residue 16 of a β₂ adrenergic receptor, the method comprising the steps of:
measuring a baseline pulmonary response for each of a plurality of subjects, each subject having the β₂ adrenergic receptor;
treating each subject with a pair of treatment agents, each agent being administered separately for a clinically relevant period in a randomized crossover treatment sequence, the agents being the inhaled corticosteroid and the leukotriene receptor antagonist;
determining a change in response from the baseline pulmonary response for each subject after each treating step, where a positive pulmonary response is an increase from the baseline and a negative pulmonary response is a response that is not a positive pulmonary response;
assigning each subject to one of a plurality of groups defined by genotype that encodes amino acid 16 of the B2AR gene, at least one group containing subjects homozygous for arginine;
plotting a regression plot between responses to the treatment agents for each group;
identifying in the regression plot a negative correlation between the pulmonary response to the treatment agents in the arginine-homozygous subjects, wherein said homozygous subjects exhibit a positive pulmonary response to the therapy with the inhaled corticosteroid and a negative pulmonary response to the therapy with the leukotriene receptor antagonist.

11. A method as claimed in claim 10 wherein the measured pulmonary response is forced expiratory volume in one second (FEV₁).

12. A method as claimed in claim 11 wherein the positive pulmonary response is an increase of at least about 7.5% of the baseline pulmonary response.

13. The method as claimed in claim 10 wherein the inhaled corticosteroid is selected from the group consisting of fluticasone propionate, beclomethasone dipropionate, budesonide, triamcinalone acetonide, flunisolide, ciclesonide, and mometasone.

14. The method as claimed in claim 10 wherein the leukotriene receptor antagonist is selected from the group consisting of montelukast sodium, zafirlukast and pranlukast.

15. The method as claimed in claim 10 wherein the inhaled corticosteroid is fluticasone propionate and the leukotriene receptor antagonist is montelukast sodium.
